(19) United States
(12) Patent Application Publication
(10) Pub. No.: US 2005/0043209 A1
(43) Pub. Date: Feb. 24, 2005

(54) Sustained release fragrance matrix and methods

(76) Inventors: Peter Schmiedel, Dusseldorf (DE); Axel Becker, Pfungstadt (DE); Ingo Hardacker, Hamminkeln (DE); Andreas Ferencz, Dusseldorf (DE)

Correspondence Address: WOODCOCK WASHBURN LLP, ONE LIBERTY PLACE, 46TH FLOOR, PHILADELPHIA, PA, 19103, US

(21) Appl. No.: 10/871,233

(22) Filed: Jun. 18, 2004

Related U.S. Application Data
(63) Continuation of application No. PCT/EP02/14050, filed on Dec. 11, 2002.

(30) Foreign Application Priority Data
Dec. 20, 2001 (DE) 10163142.1

(51) Int. Cl. A61K 7/46

(52) U.S. Cl. 512/1

(57) ABSTRACT

The present invention relates to fragrance- or odorant-laden polymer capsules and processes for producing the same.

### CROSS REFERENCE TO RELATED APPLICATIONS

This application is a continuation of PCT/EP02/14050, filed Dec. 11, 2002, which claims the benefit of DE 10163142.1, filed Dec. 20, 2001, the complete disclosures of which are hereby incorporated by reference in their entirety.

### FIELD OF THE INVENTION

The present invention relates to fragrance- or odorant-laden polymer capsules and processes for producing the same.

### BACKGROUND

For many products, it is currently attractive for esthetic reasons to apply active ingredients, for example fragrances and odorants or oils such as essential oils and aroma oils, perfume oils, care oils or silicone oils, for example, in cosmetic products or in laundry detergents and cleaning compositions. Such ingredients frequently lose their activity even in the course of storage or else directly when they are used. Some of these substances may also have insufficient stability for use or cause disruptive interactions with other product constituents. It is therefore of interest to control such substances and to use them with maximum effectiveness at the desired point of use.

Moreover, there is the frequent problem for example in the case of cleaning compositions, for example all-purpose cleaners, bathroom cleaners and especially floor cleaners, that their fragrance is very intense as they are used or immediately thereafter, but the fragrance is barely perceptible any more after a short time. There are similar problems in the case of textiles after washing: the initially intense fragrance subsides relatively rapidly.

For this reason, the active substances are often added to the products in spatially delimited, protected form. For example in the form of capsules, spheres, drops or as a second phase. In addition to the esthetic advantages, such spatial delimitations in many cases also exhibit stability and formulation advantages. Frequently, sensitive substances are enclosed in capsules of various sizes, adsorbed on suitable carrier materials or chemically modified. The release may then be activated with the aid of a suitable mechanism, for example mechanically by shearing, or by diffusion directly out of the matrix material. Also often desirable are systems which are suitable as encapsulation, transport or administration vehicles, often also referred to synonymously as delivery systems or carrier systems.

There already exist numerous commercial delivery systems which are based on porous polymer particles or liposomes (for example Microsponges® from Advanced Polymer Systems or else Nanotopes® from Ciba-Geigy; on this subject, see B. Herzog, K. Sommer, w. Baschong, J. Röding “Nanotopes™: A Surfactant Resistant Carrier System” in SÖFW-Journal 124, 10/98, pages 614 to 623). However, the disadvantage of these conventional, prior art delivery systems is that they only have a small loading potential, the particle size of the polymer spheres is usually in the range from a few micrometers to a few 100s of μm, and encapsulation of the active substances generally cannot be effected in situ. The modification of the capsule surfaces is impossible or very complicated. Liposomes also have inadequate stability for many applications. A further disadvantage of these conventional systems, is that release of active substances at the site of the specific application frequently cannot be controlled.

In addition, the production of fragrance- and/or odorant-laden capsules in accordance with the prior art often also introduces troublesome or toxic, malodorous or aggressive constituents into the formulation. Often, the encapsulations are carried out under aggressive conditions which stress the active ingredients to be encapsulated (high temperatures, long reaction times, occurrence of free radicals, etc.).

EP 0 397 245 A2 describes a process for producing perfume particles in which a polymeric support material in the melt is admixed with a perfume and the resulting melt is solidified by cooling with liquid nitrogen. The solid which forms is subsequently ground until the perfume particles have the desired size. One disadvantage of this process is the restriction to support materials which melt in a range compatible with the perfume. Owing to the melting operation in the production, the resulting perfume particles have a high density, which, owing to the compact matrix of polymeric support material, only enables low, uncontrollable release of the perfume. In addition, only low loading with perfumes is possible owing to the compact matrix. A modification of the polymeric support material to influence the substantivity of the capsules is possible only at great cost and convenience by modifying the polymer material, and is also restricted by the requirement for meltability at relatively low temperatures.

The German patent DE 35 38 429 C2 describes a process for producing a flavor-masked powder with controlled release for use in edible pharmaceutical compositions. To produce the capsules, a polymer is dissolved in a solvent, admixed with an active ingredient and dispersed. The solvent is subsequently removed again. The powders produced by this process are not suitable for use in laundry detergents and cleaning compositions.

A process for preparing polymer capsules laden with pharmaceuticals is disclosed by R. Bodmeier et al., “Spontaneous formation of drug-containing acrylic nanoparticles”, J. Microencapsulation, 1991, Vol. 8, No. 2, pages 161 to 170. In this process, the pharmaceutical is dissolved in a water-miscible organic solvent, admixed with a specifically ammonium-functionalized acrylic polymer (Eudragit® RS or RL 100) and transferred into water. This forms a desired dispersion of polymer capsules in the organic solvent. However, this process is restricted to the use of the specific ammonium-functionalized acrylic polymers, because only these polymers have precisely the required ionic character with simultaneous water insolubility in order to prevent clumping of the polymer capsules.

### SUMMARY

It is thus an object of the present invention to provide a matrix or depot system in the form of polymer capsules having properties improved over the prior art, which is suitable especially for encapsulating fragrant components, and also appropriate production processes.

It is a further object of the present invention to provide a system which, especially when used in laundry detergents or cleaning compositions, generates a long-lasting fragrance without the fragrance being unpleasantly intense immediately after the use of the product.

It is a further object of the present invention to provide fragrance capsules having sustained release, especially of fragrant and/or odorant substances. These fragrant capsules should be especially suitable for use in laundry detergents and cleaning compositions.

### DETAILED DESCRIPTION

A first embodiment of the inventive polymer capsules provides fragrance- or odorant-laden matrix and/or depot systems which comprise at least one polymeric support or matrix material based on especially water-insoluble celluloses and/or cellulose derivatives which have average molecular weights of more than 30 000, preferably more than 35 000, more preferably more than 40 000, the polymer capsules also comprising at least one fragrant component. This fragrant component may in particular be selected from the group of fragrances and/or odorants, fragrance and/or odorant formulations and oils such as essential oils, aroma oils, perfume oils, care oils and silicone oils. In addition, these polymer capsules comprise at least one surfactant. If the polymer material used is a cellulose derivative, this may be selected, for example, from the group of alkylcelluloses, especially ethylcellulose, and cellulose esters, especially cellulose acetate and cellulose acetate butyrate.

A second embodiment of the inventive polymer capsules provides fragrance- or odorant-laden matrix and/or depot systems which comprise at least one polymeric support or matrix material substantially based on water-insoluble polymers having average molecular weights of more than 30 000, especially more than 35 000, preferably more than 40 000. These polymer capsules comprise at least one fragrant component which may preferably be selected from the group of fragrances and/or odorants, fragrance and/or odorant formulations and oils such as essential oils, aroma oils, perfume oils, care oils and silicone oils. In addition, these polymer capsules comprise at least one surfactant. The polymer may be a nonionic or an ionic polymer. The polymer may additionally include a blocked and/or random comonomer. The polymer may in particular be selected from the group of polyalkylenes such as polybutadienes, poly(butadiene-co-acrylonitriles), polyisobutenes, polyamides, polystyrenes, polyisoprenes, polycarbonates, polyesters, polyacrylates, polymethacrylates, polyurethanes, polydimethyl itaconates, polydiamyl fumarates, polybenzyl vinyl ethers, polyvinyl alcohols, polyallyl alcohols, polyvinyl formals, polyvinyl butyrals, poly(2-vinyl-4,7-dihydro-1,3-dioxepines), polyvinyl methyl ketones, polymethyl isopropyl ketones, polyvinyl pivalates, polyvinyl acetylacetates, poly(p-formylstyrenes), poly(2-vinylpyridines), polydimethylfulvenes, poly(carbonyl-1-furfuryltrimethylenes), polytetrahydrofurans, polyglutaraldehydes, polyoxycarbonyloxyhexa-methylenes, polyethylene adipates, polyvinyl alcohol acetates, polyvinyl butyral acetals, polylactic acids and polyoxymethylhexadecylsilylenes. The polymer may be ionically modified. This ionic modification is preferably effected by ionic groups (for example ammonium or carboxylate functions), by grafting or by copolymerization with suitable ionic comonomers. The polymer may also be an ionic polymer which simultaneously optionally constitutes or includes the surfactant (for example Eudragit® RS and RL 100); this means in particular that the function of the surfactant is thus integrated into the polymer, i.e. the ionic polymer brings the ionic charge into the system.

A third embodiment of the inventive polymer capsules provides fragrance- or odorant-laden matrix and/or depot systems which comprise at least one polymeric support or matrix material based on an ionic polymer. In general, the average molecular weights are more than 30 000, preferably more than 35 000, more preferably more than 40 000. The polymer capsules additionally comprise at least one fragrant component which may be selected from the group of fragrances and/or odorants, fragrance and/or odorant formulations and oils such as essential oils, aroma oils, perfume oils, care oils and silicone oils. These polymer capsules may also comprise at least one surfactant. The polymer may include a blocked or random comonomer. In addition, the polymer may be an ionic polymer or ionically modified. This modification may be effected, for example, by ionic groups, especially ammonium and/or carboxylate functions, by grafting and/or by copolymerization with suitable ionic comonomers. In a preferred variant of the third embodiment, the ionic polymer may be an ammonium-functionalized (meth)acrylate copolymer, preferably a poly(ethylene-methyl methacrylate-trimethylammonioethyl methacrylate) copolymer, in which case the stoichiometric ratios of the individual monomer units in the poly(ethylene-methyl methacrylate-trimethylammonioethyl methacrylate, copolymer may be 1:2:0.1 or 1:2:0.2 (for example Eudragit® RS and RL 100).

Useful fragrances or odorants and/or fragrance or odorant formulations for all three embodiments of the inventive polymer capsules are natural or synthetic fragrances and/or odorants or fragrance or odorant formulations. The fragrances or odorants used may in particular be perfume oils or mixtures of perfume oils which are typically used in laundry detergents and cleaning compositions or cosmetics. For instance, the perfume oils or fragrances used may be individual fragrance compounds, for example the synthetic products of the ester, ether, aldehyde, ketone, alcohol and hydrocarbon type. Examples of ester-type fragrance compounds include benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethyl methylphenylglycinate, allyl cyclohexylpropionate, styrallyl propionate, benzyl salicylate, cyclohexyl salicate, floramate, melusate and jasmacyclate. Mention should also be made here of the esters of fragrant alcohols with inorganic acids or organic acids, as disclosed in the prior art. The ethers include, for example, benzyl ethyl ether and ambroxan, the aldehydes include, for example, linear alkanals having from 8 to 18 carbon atoms, citral, citronellal, citronellyloxy-acetaldehyde, cyclamen aldehyde, hydroxy citronellal, lilial and bourgeonal, and the ketones include, for example, the ionones, α-isomethylionone and methyl cedryl ketone. The alcohols include anethole, citronellol, eugenol, geraniol, linalool, phenylethyl alcohol and terpineol, and the hydrocarbons include mainly the terpenes such as limonene and pinene. Preference is given to using mixtures of different fragrances which are matched to each other in such a way that together they generate a pleasing fragrance note. Suitable perfume oils may also comprise natural fragrance mixtures as obtainable from vegetable sources, for example pine oil, citrus oil, jasmine oil, patchouli oil, rose oil or ylang-ylang oil. Likewise suitable are clary sage oil, chamomile oil, oil of cloves, balm oil, mint oil, cinnamon leaf oil, lime blossom oil, juniperberry oil, vetiver oil, olibanum oil, galbanum oil and labdanum oil, and also orange blossom oil, neroliol, orange peel oil and sandalwood oil. Natural fragrant components which should also be mentioned include: extracts from blossoms (lily, lavender), stems and leaves (geranium, petitgrain), fruits (aniseed, coriander, cumin, nutmeg), fruit peels (bergamot, lemon, oranges), roots (maize, angelica, celery, cardamom, costas, iris, calmus, vetiver), woods (guaiac, cedarwood, rosewood), herbs and grasses (tarragon, lemongrass, thyme), needles and branches (spruce, fir, pine, dwarf-pine), resins and balsams (galbanum, elemi, benzoin, myrrh, olibanum, opoponax, labdanum). Also useful are animal raw materials, for example civet and castoreum. Further compounds which may also be used as fragrances are: nitriles, sulfides, oximes, acetals, ketals, acids, Schiff bases, heterocyclic nitrogen compounds (indole, quinoline), pyrazines, (anthranilic) amines, amides, organohalogen compounds (rose acetate), nitrated compounds (nitromusk), heterocyclic sulfur compounds (thiazoles) and heterocyclic oxygen compounds (epoxides). Examples of perfume oils which are used with particular preference in laundry detergents and cleaning compositions and cosmetics are: orange oil, especially sweet orange oil, geraniol, rose oil, lilial, α-hexylcinnamaldehyde, Iso E Super, α-amylcinnamaldehyde, propidyl, hexyl salicylate, Cedramber, myraldyl acetate, PTBCA 25 cis (cyclohexanol, 4-(1,1-dimethylethyl) acetates), linalool, tetrahydrolinaool, citronellol, Aldehyde C12, troenan, floramate, diphenyl ether, Isoraldein 70, cyclohexyl salicylate, sandelice, boisambrene forte. For further details relating to fragrances or odorants and/or fragrance or odorant formulations, reference is made to P. Kraft et al., “Allerlei Trends: die neuesten Entwicklungen in der Riechstoffchemie” [All kinds of trends: the most recent devlopments in fragrance chemistry] in Angew. Chem. 2000, 112, 3106-3138, whose contents are hereby incorporated by reference.

The inventive polymer capsules of all three embodiments may additionally comprise water. Their average particle size may vary within wide ranges; the average particle size may generally be from about 50 nm to about 500 μm, especially from about 100 nm to about 250 μm, preferably from about 200 nm to about 100 μm. The content of fragrant component(s) in the inventive polymer capsules of all three embodiments may also vary within wide ranges; it is generally from 1% by weight to 60% by weight, preferably from 10% by weight to 50% by weight, these weight data being based on the polymer capsules. The content of surfactant in the inventive polymer capsules of all three embodiments may likewise vary within wide ranges; it is generally from 0.01% by weight to 50% by weight, but preference is given to a surfactant content of from 0.1% by weight to 40% by weight, the weight data being based on the inventive polymer capsules of the particular embodiment.

According to the invention, polymer capsules refer in particular to depot or matrix capsules in the form of liquid-rich disperse systems composed of at least one polymer and at least one fragrant component. According to the invention, the term capsules does not mean core/shell structures, but rather matrix or depot particles. These inventive matrix and/or depot structures do not consist of a conventional core/shell structure, but rather of a system which may be formed owing to physical and/or chemical interactions, for example network formation. The inventive capsules are in particular dimensionally stable, readily deformable particles. In the present case, these polymer capsules in all three embodiments may form particulate, especially spongelike structures composed of polymer and fragrant component. The fragrant component may in particular be present in homogeneous distribution through the polymer. Depending on the compatibility of fragrant component on the one hand and polymer on the other, monophasic or biphasic capsule systems are obtained. When, for example, a fragrant component is used which is incompatible with the polymer, application of the capsules to films results in a cloudy, microscopically biphasic film. When, in contrast, the fragrant component is compatible or miscible with the polymer, application of the capsules to films results in a clear, homogeneous, microscopically monophasic film, i.e. the fragrant component is then incorporated homogeneously into the polymer like a plasticizer.

The polymers and/or fragrant component(s) present in all three embodiments of the inventive polymer capsules are in particular substantially water-insoluble or at least only sparingly soluble in the aqueous phase. In such a case, the polymers and/or the fragrant components are preferably soluble to a degree of less than 10%, preferably of less than 5%, in particular of less than 1%. In the context of the present invention, aqueous phase also means those systems which may contain considerable amounts of organic solvents, although the water content should not be less than 50%.

The inventive polymer capsules according to all three embodiments may comprise ionic, i.e. cationic or anionic, or nonionic surfactants (interface-active substances). Depending on the desired use of the polymer capsules, different surfactants are used in order to control the substantivity of the resulting capsules or to tailor for the particular application. For example, cationic surfactants in particular are used for the inventive polymer capsules for use in fabric softeners, since positively charged or cationically modified capsules attach readily to the negatively charged fibers of garments and thus have good substantivity toward these fibers. Cationic surfactants suitable in accordance with the invention may in particular be selected from the group of quaternary ammonium compounds such as dimethyldistearylammonium chloride (CTMA-CI), dodecyltrimethylammonium bromide, didodecyldimethylammonium bromide, tridodecylmethylammonium bromide; tetradodecylammonium bromide; ester quats, especially quaternized fatty acid trialkanolamine ester salts; salts of long-chain primary amines of quaternary ammonium compounds such as hexadecyltrimethylammonium chloride; cetrimonium chloride or lauryldimethylbenzylammonium chloride. In contrast, anionic surfactants are preferentially suitable for use in the inventive polymer capsules for use in laundry detergents and cleaning compositions. Anionic surfactants suitable in accordance with the invention may in particular be selected from the group of soaps; alkylbenzenesulfonates; alkanesulfonates; olefinsulfonates; alkyl ether sulfonates; glycerol ether sulfonates; α-methyl ester sulfonates; sulfo fatty acids; alkyl sulfates such as sodium dodecyl sulfate (SDS); fatty alcohol ether sulfates; glycerol ether sulfates; fatty acid ether sulfates; hydroxy mixed ether sulfates; monoglyceride (ether) sulfates; fatty acid amide (ether) sulfates; mono- and dialkyl sulfosuccinates; mono- and dialkyl sulfosuccinamates; sulfotriglycerides; amide soaps; ether carboxylic acids and their salts; fatty acid isethionates; fatty acid sarcosinates; fatty acid taurides; N-acylamino acids such as acyl lactylates, acyl tartrates, acyl glutamates and acyl aspartates; alkyloligoglucoside sulfates; protein fatty acid condensates, especially vegetable products based on wheat; alkyl (ether) phosphates. Especially in the case of the use of ionic polymers in the inventive polymer capsules, a nonionic surfactant may also be present in the polymer capsules. In principle, it is also possible when using nonionic polymers to use nonionic surfactants, especially in addition to ionic surfactants. The nonionic surfactants may be selected, for example, from the group of (i) nonpolymeric nonionic surfactants such as alkoxylated, preferably ethoxylated, fatty alcohols, alkylphenols, fatty amines and fatty acid amides; alkoxylated triglycerides, mixed ethers and mixed formals; optionally partially oxidized alk(en)yloligoglycosides; glucuronic acid derivatives; fatty acid N-alkylglucamides; protein hydrolyzates, especially alkyl-modified protein hydrolyzates; low molecular weight chitosan compounds; sugar esters; sorbitan esters; amine oxides; and (ii) polymeric nonionic surfactants such as fatty alcohol polyglycol ethers; alkylphenol polyglycol ethers; fatty acid polyglycol esters; fatty acid amide polyglycol ethers; fatty amine polyglycol ethers; polyol fatty acid esters; polysorbates. The selection and combination of ionic or nonionic polymers with charged, i.e. anionic or cationic, or uncharged, i.e. nonionic, surfactants may provide “tailored” polymer capsules which have an exact charge or substantivity required for the desired application.

The inventive polymer capsules according to all three embodiments may be carriers of a surface charge. Therefore, the polymer capsules may also have a zeta potential. The zeta potential is the externally active potential of the particles which is responsible for their electrokinetic phenomena and is therefore also referred to as the electrokinetic potential. The zeta potential can be determined in particular by microscopic observation of the electrophoretic migration of suspended particles. In general, this zeta potential in the state dispersed in aqueous media is greater than 10 mV, preferably greater than 20 mV, more preferably greater than 30 mV. This surface charge may be advantageous for the substantivity of the inventive polymer capsules on substrates, for example fibers of clothing.

The present invention also provides production processes for the inventive polymer capsules. It is possible to differentiate between the embodiment of the emulsification process (first embodiment) and the precipitation process (second embodiment).

In the first embodiment, the inventive polymer capsules are produced by the emulsification process. This process for preparing fragrance and/or odorant-laden matrix and/or depot systems in the form of polymer capsules which contain at least one fragrant component, as described above, is characterized by the following process steps:

(a) providing a preferably homogeneous mixture, especially solution or dispersion, which comprises at least one polymer and at least one fragrant component, especially selected from the group of fragrances and/or odorants, fragrance and/or odorant formulations and oils such as essential oils, aroma oils, perfume oils, care oils and silicone oils, with a substantially water-immiscible organic solvent or dispersant, especially alkanes such as pentane, hexane (n-hexane or cyclohexane) or ethyl methyl ketone and ethyl acetate;

(b) introducing the mixture prepared under (a) into water or into an aqueous solution, or vice versa;

(c) preparing an emulsion of the mixture prepared under (b) in the presence of at least one suitable emulsifier, especially surfactant;

(d) removing the organic solvent from the emulsion prepared under (c), so as to obtain fragrance-laden polymer capsules in aqueous dispersion;

(e) optionally removing the polymer capsules obtained in this way.

In this first embodiment, the mixture prepared under (a) may be introduced into water or into an aqueous solution with stirring and/or heating. Equally, water or an aqueous solution may also be introduced with stirring and/or heating into the mixture prepared under (a). The preparation of the emulsion carried out in step (c) may be effected by the action of shear forces. This may involve, for example, high-pressure homogenization or the use of Ultraturrax stirrers or disperser disks. The content of surfactants in the emulsion prepared in step (c) may vary within wide ranges: the surfactant content is generally up to 10% by weight, the weight data being based on the emulsion. It is crucial that a sufficient content of surfactant, to be determined in the individual case, is used, so that repulsion of the particles or droplets which form is achieved. At the same time, the content of surfactant must not be too high, so that there is dissolution of the polymer. The content of polymer in the emulsion prepared in step (c) may also vary within wide ranges; it is generally up to 20% by weight, preferably up to 10% by weight, these weight data also being based on the emulsion. The content of fragrant component in the emulsion prepared in step (c) may likewise vary within wide ranges: it is generally up to 10% by weight, the weight data being based on the emulsion. The removal of the solvent or dispersant used, carried out in step (d), may be effected by methods known per se and familiar to those skilled in the art. In particular, this involves the removal of the solvent or dispersant under reduced pressure, preferably in a rotary evaporator. A possible alternative is also an evaporation, especially under atmospheric pressure, for which volatile organic solvents and dispersants, for example n-pentane, n-hexane or cyclohexane, are preferentially suitable. Especially in the case of the use of intensely odorous solvents (for example ethyl acetate), the removal or separation of the solvent carried out in step (d) should be substantially quantitative, so that the action of the fragrant component is not impaired by solvent still present. Solvents which have a low degree of water miscibility or which are very volatile, for example alkanes, are therefore more suitable in this regard.

In a particularly preferred variant of the first embodiment, the procedure in the inventive emulsification process may be as follows: a solution of a fragrance and of a polymer is prepared in a water-immiscible or only slightly water-miscible organic solvent or dispersant. This solution or dispersion is subsequently emulsified in water in the presence of suitable emulsifiers and apparatus (Ultraturrax, disperser disk, high-pressure homogenizer, etc.) to form a fine emulsion. Subsequently, appropriately under reduced pressure or with the aid of a rotary evaporator, the solvent or dispersant is removed from this emulsion. An odorant-containing dispersion remains which is filmable on surfaces and generates a relatively long-lasting fragrance impression. The polymers which can be used here are all of the aforementioned polymers which are soluble in appropriate solvents and dispersants but insoluble in water. The organic, water-immiscible solvent or dispersant should have maximum volatility, so that it can be removed readily without significant amounts of the fragrance also escaping. Particularly suitable are, for example, ethyl acetate, short-chain hydrocarbons, for example pentane or hexane, or ethyl methyl ketone. After the removal of the solvent or dispersant, a aqueous dispersion of the inventive particles is obtained. The inventive particles or particle dispersions, especially those stabilized cationically, are particularly suitable for use in washing aftertreatment compositions or fabric softeners. In the last rinse of a washing cycle, they attach at least partly to the textiles and bring about an initially relatively weak fragrance of the textiles which becomes more intense after a few hours to days. Further details are described in the working examples. The inventive particles or particle dispersions are equally particularly suitable for use in floor cleaners. These often contain film-forming systems for sealing floors or for application of a shiny, polishable film. Suitable for this purpose are, for example, acrylate dispersions or else wax dispersions, for example beeswax or carnauba wax. The fragrance-containing microparticles can also be integrated into these films and may in this way generate a fragrance impression lasting over several days in the room in which the inventive composition is applied. It will be appreciated that the use of the inventive compositions is not restricted to floor cleaners. They may likewise be used in kitchen, bathroom or all-purpose cleaners.

In a second embodiment, the fragrant polymer capsules are prepared by the precipitation process. This process for preparing fragrance- and/or odorant-laden matrix and/or depot systems in the form of polymer capsules which comprise at least one fragrant component, as described above, is characterized by the following process steps:

(a) providing a preferably homogeneous mixture, especially solution or dispersion, which comprises at least one polymer and at least one fragrant component, especially selected from the group of fragrances and/or odorants, fragrance and/or odorant formulations and oils such as essential oils, aroma oils, perfume oils, care oils and silicone oils, with a substantially water-miscible organic solvent or dispersant, especially acetone or alcohol;

(b) introducing the mixture prepared under (a) into water or into an aqueous solution, preferably with a dropwise addition and/or stirring, in the presence of a suitable surfactant, so as to obtain fragrance-laden polymer capsules in aqueous dispersion;

(c) optionally removing the polymer capsules obtained in this way.

In this second embodiment, the mixture prepared under (a), especially with stirring and/or optionally with heating, may be introduced slowly into water or into the aqueous solution. The introduction of the mixture prepared under (a) is preferably effected dropwise. This achieves the formation of a finely disperse precipitate of the inventive polymer capsules. When the mixture prepared under (a) is added to water or the aqueous solution too rapidly, the polymer capsules precipitate out as a large, coherent plastic mass or in the form of larger particles of flocs.

In a particularly preferred variant of the second embodiment, the procedure in the inventive precipitation process may be as follows: a fragrance is dissolved together with a polymer in a water-miscible organic solvent, for example alcohol or acetone. The polymer should not be water-soluble. This solution is now introduced into a sufficient amount of water, in the course of which the polymer precipitates out and encloses the fragrance. The dispersion which forms is filmable, optionally with the addition of assistants or further film formers, or can be further processed in another way. A prerequisite for this process is that the polymer precipitates in finely dispersed form on introduction into the precipitant. This is only the case for very few polymers, i.e. for those which bear ionic groups to such an extent that although they are water-insoluble, they nevertheless generate electrostatic repulsion of the precipitate seeds, so as to form a finely dispersed precipitate. An example is the aforementioned Eudragit® RS or RL (Röhm). However, most polymers precipitate out in the course of the precipitation in water as a large, coherent plastic mass or in the form of larger particles or flocs and therefore cannot be used as such alone. However, ionic surfactants may then be added to the system in accordance with the invention. The surfactants may be added to the aqueous precipitation bath and/or to the organic mixture or dispersion, as long as they are soluble therein. The surfactants bring about electrostatic repulsion of the seeds formed in the precipitation and thus bring about a finely dispersed precipitate. The inventive addition of surfactant(s) thus increases the number of polymers suitable for this process enormously. In the presence of sufficient concentrations of ionic surfactant, it is possible in principle to use all polymers which are insoluble in water but soluble in water-miscible solvents. Suitable are, for example, water-insoluble cellulose ethers and esters such as ethylcellulose, nitrocellulose, cellulose acetate, cellulose acetate butyrate, polyvinyl acetate, water-insoluble vinyl acetate/vinylpyrrolidine copolymers and also various acrylate or methacrylate polymers, for example Ultrahold® 8 (acrylate/acrylamide copolymer) from BASF. Polyurethane-based polymers may also be used directly as long as they have the correct solubility characteristics.

Further examples of suitable polymers, i.e. those which are insoluble in water but soluble in water-miscible solvent, can be found in the following listing: poly-1,3-butadienes (T); poly(butadiene-co-acrylonitriles, (A); polyisobutenes (T); generally polyacrylates (T), apart from, for example, poly(2-hydroxyethyl acrylate) which is water-soluble; many polymethacrylates (T), for example isotactic and syndiotactic polymethyl methacrylates (T,A), polydimethyl itaconates (A,T); polydiamyl fumarates (T); polybenzyl vinyl ethers (A); polyvinyl alcohol acetyls (A); polyallyl alcohol (T); polyvinyl formals (AA); polyvinyl butyral acetals (E) poly(2-vinyl-4,7-dihydro-1,3-dioxepines) (A); polyvinyl methyl ketones (A, AA, T); polymethyl isopropenyl ketones (A); polyvinyl pivalates (A); polyvinyl acetylacetates (AA, T); atactic polystyrenes (T); poly(p-formylstyrenes) (T); poly(2-vinylpyridines) (A); polydimethylfulvenes (A); poly(carbonyl-1-furfuryltrimethylenes) (A); polytetrahydrofuran (T); polyglutaraldehydes (T); poly(oxycarbonyloxyhexamethylenes) (A); polyethylene adipates (T); polylactic acids (A), poly(oxymethylhexadecylsilylenes) (T). Abbreviations (A), (E), (T) and (AA) designate the water-miscible organic solvent in which the polymers are soluble, wherein acetone is (A), ethanol is (E), tetrahydrofuran is (T), and acetic acid is (AA).

Preference is given to polymers soluble in acetone and alcohols, for example ethanol, over polymers which are soluble in tetrahydrofuran or acetic acid.

In the process according to the invention, both in the first and in the second embodiment, the mixture can be prepared in step (a) by adding the polymer and the fragrant component to the organic solvent or dispersant. This addition may preferably be effected under stirring and optionally with heating, with the proviso that as far as possible a homogeneous mixture is formed.

In the processes according to the invention, both in the first and in the second embodiment, the optional removal of the fragrance-laden polymer capsules may be effected by methods known to those skilled in the art. These are in particular spray drying under gentle conditions, ultrafiltration, dialysis or freeze-drying (lyophilization). In the case of relatively large particles, removal may also be achieved by centrifugation.

The surfactant (interface-active substance) used in the process according to the invention both in the first and in the second embodiment may be an ionic or nonionic surfactant. However, it is crucial that the resulting polymer capsules have a surface charge. This charge prevents agglomeration of the polymer capsules with one another during the inventive production processes in both embodiments. This charge may also be achieved by the use of charged surfactants; alternatively, it is also possible to use charged polymers. However, when oppositely charged constituents are used, i.e., for example, polymer or surfactant, there is the risk that complexing of the constituents will occur in the production process, from which the formation of larger particles results. Preference is therefore given to the use of surfactants and polymers having the same charge. Alternatively, charge reversal of charged polymers may also be effected by oppositely charged surfactants; this makes it possible to form “tailored” polymer capsules.

When an anionic surfactant is used in the processes according to the invention, both in the first and in the second embodiment, this may be selected, for example, from the group of soaps; alkylbenzenesulfonates; alkanesulfonates; olefinsulfonates; alkyl ether sulfonates; glycerol ether sulfonates; α-methyl ester sulfonates; sulfo fatty acids; alkyl sulfates such as sodium dodecyl sulfate (SDS); fatty alcohol ether sulfates; glycerol ether sulfates; fatty acid ether sulfates; hydroxy mixed ether sulfates; monoglyceride (ether) sulfates; fatty acid amide (ether) sulfates; mono- and dialkyl sulfosuccinates; mono- and dialkyl sulfosuccinamates; sulfotriglycerides; amide soaps; ether carboxylic acids and their salts; fatty acid isethionates; fatty acid sarcosinates; fatty acid taurides; N-acylamino acids such as acyl lactylates, acyl tartrates, acyl glutamates and acyl aspartates; alkyloligoglucoside sulfates; protein fatty acid condensates, especially vegetable products based on wheat; alkyl (ether) phosphates. When, however, a cationic surfactant is used, this may be selected, for example, from the group of quaternary ammonium compounds such as dimethyldistearylammonium chloride (CTMA-CI), dodecyltrimethylammonium bromide, didodecyldimethylammonium bromide, tridodecylmethylammonium bromide, tetradodecylammonium bromide; ester quats, especially quaternized fatty acid trialkanolamine ester salts; salts of long-chain primary amines of quaternary ammonium compounds such as hexadecyltrimethylammonium chloride; cetrimonium chloride or lauryldimethylbenzylammonium chloride. Especially when ionic polymers are used, a nonionic surfactant may be present in the polymer capsules; in this case, the nonionic surfactant may be selected, for example, from the group of (i) nonpolymeric nonionic surfactants such as alkoxylated, preferably ethoxylated, fatty alcohols, alkylphenols, fatty amines and fatty acid amides; alkoxylated triglycerides, mixed ethers and mixed formals; optionally partially oxidized alk(en)yloligoglycosides; glucuronic acid derivatives; fatty acid N-alkylglucamides; protein hydrolyzates, especially alkyl-modified protein hydrolyzates; low molecular weight chitosan compounds; sugar esters; sorbitan esters; amine oxides; and (ii) polymeric nonionic surfactants such as fatty alcohol polyglycol ethers; alkylphenol polyglycol ethers; fatty acid polyglycol esters; fatty acid amide polyglycol ethers; fatty amine polyglycol ethers; polyol fatty acid esters; polysorbates.

The polymer used in the processes according to the invention, both in the first and in the second embodiment, may be a nonionic or an ionic polymer. The polymer may likewise include a blocked and/or random comonomer. In particular, the polymer may be selected from the group of celluloses and cellulose derivatives such as ethylcelluloses, cellulose acetates and cellulose acetate butyrates, polyalkylenes such as polybutadienes, poly(butadiene-co-acrylonitriles), polyisobutenes, polyamides, polystyrenes, polyisoprenes, polycarbonates, polyesters, polyacrylates, polymethacrylates, especially ammonium-functionalized poly-methacrylates, polyurethanes, polydimethyl itaconates, polydiamyl fumarates, polybenzyl vinyl ethers, polyvinyl alcohols, polyallyl alcohols, polyvinyl formals, polyvinyl butyrates, poly(2-vinyl-4,7-dihydro-1,3-dioxepines), polyvinyl methyl ketones, polymethyl isopropyl ketones, polyvinyl pivalates, polyvinyl acetylacetates, poly(p-formylstyrenes), poly(2-vinylpyridines), polydimethylfulvenes, poly(carbonyl-1-furfuryltrimethylenes), polytetrahydrofurans, poly(glutaraldehydes), polyoxycarbonyloxyhexamethylenes, polyethylene adipates, polyvinyl alcohol acetates, polyvinyl butyral acetals, polylactic acids and polyoxymethylhexadecylsilylenes.

When the polymer used in the processes according to the invention, both in the first and in the second embodiment, is an ionic polymer, this may simultaneously constitute or include the surfactant. This means in particular that the function of the surfactant is thus integrated in the polymer, i.e. the ionic polymer brings the ionic charge into the system. It is thus the ionic character of the polymer that actually ensures the desired electrostatic repulsion of the polymer capsules. In addition, the polymer may also be ionically modified. This modification may be effected, for example, by ionic groups, especially ammonium and/or carboxylate functions, by grafting and/or by copolymerization with suitable ionic comonomers. In a preferred variant of both embodiments, the ionic polymer may be an ammonium-functionalized (meth)acrylate copolymer, preferably a poly(ethylene-methyl methacrylate-trimethylammonioethyl methacrylate) copolymer, in which case the stoichiometric ratios of the individual monomer units in the poly(ethylene-methyl methacrylate-trimethylammonioethyl methacrylate) copolymer may be 1:2:0.1 or 1:2:0.2 (for example Eudragit® RS and RL 100).

The polymers and/or fragrant components used in both embodiments of the processes according to the invention should preferably be substantially water-insoluble or at least only sparingly soluble in the aqueous phase. In such a case, the polymers and/or the fragrant component(s) should be soluble in the aqueous phase to a degree of less than 10%, preferably of less than 5%, especially of less than 1%.

The average particle size of the polymer capsules obtainable by the processes according to the invention in both embodiments may vary within wide ranges: it is generally from about 50 nm to about 500 μm, especially from about 100 nm to about 250 μm, preferably from about 200 nm to about 100 μm. Control of the particle size is possible by variation of the process parameters (for example variation of the surfactant content, of the overall concentration of the solution or dispersion, type and concentration of the polymer, type and concentration of the fragrant component, etc.). In the first embodiment, the emulsification process, the particle size may additionally be controlled by the energy input when forming the emulsion. In the second embodiment, the precipitation process, in contrast, the particle size may additionally be controlled especially by the droplet size and the dropwise addition rate in process step (b).

In both embodiments of the process according to the invention, the solvent or dispersant may constitute or include the surfactant. This is the case, for example, especially when oleic acid is used as the solvent or dispersant and simultaneously as the surfactant.

The present invention also provides the fragrance- and/or odorant-laden polymer capsules producible by the processes according to the invention.

The present invention further provides dispersions, preferably aqueous dispersions, which comprise the inventive polymer capsules or those producible in accordance with the invention. The content of polymer capsules in these inventive dispersions may vary within wide ranges: it is generally up to 20%, especially up to 15%, preferably up to 10%. The weight data are based on the dispersion. The inventive dispersions may be used, for example, in the form of laundry detergents and cleaning compositions, cosmetics or personal care compositions. In the case of aqueous dispersions, these may comprise varying proportions of organic solvent and dispersant (especially from the production process), although a proportion of water, based on liquid phase, of 50% should not be exceeded.

The present invention also provides the use or application of the inventive polymer capsules or of the inventive dispersions, the use and application possibilities being very numerous and extensive.

For instance, the inventive polymer capsules or those producible in accordance with the invention, and also the inventive dispersions, may be used in the laundry detergent and cleaning composition industry, or in the cosmetics and personal care industry. They may be used, for example, as a delivery system, in which case the fragrant component(s) act(s) in particular with prolonged or retarded release (known as “sustained release effect”).

The inventive polymer capsules or those producible in accordance with the invention and the inventive dispersions may also be used for controlled or retarded release of fragrant components.

A further use or application of the inventive polymer capsules or those producible in accordance with the invention and of the inventive dispersions is their application as a coating or film. The release of the fragrant component can be controlled by the selection of the type of the composition of the polymer capsules, especially by the selection of the type of the polymer and of the fragrant component(s). The application may be used, for example, to produce carrier surfaces having depot retention of fragrant components and thus serves for protection and/or for depot retention of fragrances and/or odorants, fragrance and/or odorant formulations and oils such as essential oils, aroma oils, perfume oils, care oils and silicone oils on these carrier surfaces. These inventive capsule or dispersion systems may either be filmed on hard surfaces, embedded into further film formers or else, optionally with the addition of further assistants, be attached to textiles, so that they generate a relatively long-lasting fragrance impression. According to the invention, the term “film” does not necessarily refer to a coherent, continuous layer, but rather to a distribution of the inventive polymer capsules on the surface, and this distribution may also be interrupted. The present invention thus further provides films, layers, and/or coatings which can be obtained starting from the inventive polymer capsules or dispersions.

The present invention further provides surfaces, especially surfaces of inert carrier materials, to which films, layers, coatings or the like of the aforementioned type have been applied. As described above, these are in particular surfaces having depot function for fragrant components or having controlled and/or retarded release function for fragrant components such as fragrances and/or odorants, fragrance and/or odorant compositions and oils such as essential oils, aroma oils, perfume oils, care oils and silicone oils.

The present invention is associated with a multitude of advantages. To name only a few, the following are mentioned by way of example: the invention, especially in the embodiment of the precipitation process, constitutes a process for producing fragrant polymer capsules and coatings which can be carried out with very low technical complexity and provides the inventive dispersions in ready-to-use form. The processes according to the invention thus provide, in addition to the polymer capsules themselves, equally also aqueous dispersions which can be used immediately. The use of these polymer capsules in laundry detergents, cleaning compositions or aftertreatment compositions and also cosmetics leads to a prolonged fragrance impression compared to the dosage of the pure perfume oil. Their use in cleaners, especially floor cleaners, leads to a fragrant film on the substrates and thus to a prolonged fragrance impression in the cleaned rooms. On the other hand, the fragrance impression is not as intense immediately after the application. The risk therefore falls that the consumer will find it initially to be too strong and therefore too unpleasant. In addition, the inventive dispersions are especially useful for easier dosage of fragrant compounds, especially for the formulation of laundry detergents and cleaning compositions or in the field of the cosmetics and personal care industry. For instance, these dispersions do not have to be prepared in water or aqueous solutions by subsequent dispersion of polymer capsules.

Further embodiments, modifications and variations and also advantages of the present invention can be discerned and realized by those skilled in the art when reading the description without thus leaving the framework of the present invention.

The present invention is illustrated with reference to the working examples which follow, which, however, in no way restrict the invention.

### EXAMPLES

### Example 1

Process For Producing Odorant-Containing Microparticles

An organic phase of ethyl acetate with 0.1% by weight of n-decane is prepared, into which 5% by weight of water-insoluble cellulose acetate butyrate polymer and 1% by weight of perfume oil are dissolved. With Ultraturrax® treatment, three times the amount of an aqueous emulsifier solution of 0.5% by weight of dodecyltrimethylammonium bromide is dispersed into the initially charged organic phase. This forms a finely dispersed emulsion having the aqueous phase as the outer, coherent phase. From this emulsion, the more volatile solvent is slowly drawn off under reduced pressure. A finely dispersed, milky-white dispersion having particle sizes of less than 1 μm remains. There is thus 16.6% perfume oil in the particles; the total content of perfume in the dispersion is 0.33%.

### Example 2

Precipitated Polyacrylate Particles

9.25 g of a 10% ethanolic solution (w/w) of Eudragit RS® (Röhm) are mixed with 0.75 g of perfume oil. The solution is introduced into 90 g of water with stirring. Finely dispersed polymer particles (<1 μm) having a (calculated) perfume content of approx. 45% in the particles or 0.75% in the overall dispersion precipitate out.

Owing to the cationic charge of the particles, these polymer capsules are especially suitable for use in fabric softener formulations.

### Example 3

Anionically Stabilized, Precipitated Ethylcellulose Particles

A 1% acetonic solution (w/w) of ethylcellulose (EC N7 from Hercules) is prepared, in which 0.3% of perfume oil is dissolved. The solution is introduced with stirring into ten times the amount of a surfactant-containing aqueous precipitation bath (0.02% of sodium dodecyl sulfate (SDS) in water (w/w)). The ethylcellulose precipitates out as a finely dispersed, odorant-containing precipitate (particle sizes<1 μm).

The thus prepared, anionically stabilized particles are especially suitable for formulation into anionically based cleaner formulations as described in example 12. They may either be isolated by centrifugation or filtration, or else further processed directly, for example to give a film.

The thus obtained, fine EC dispersion is applied to a glass substrate by knifecoating. After the solvent has been evaporated off, a coherent film remains which still has a fragrance even after a few days. However, more relevant in practice is the processing described in example 12 in a formulation for cleaners for hard surfaces.

### Example 4

Cationically Stabilized, Precipitated Ethylcellulose Particles

A 1% acetonic solution (w/w) of ethylcellulose (EC N7 from Hercules) is prepared, in which 0.6% of perfume oil is dissolved. This solution is introduced with stirring into the same amount of a 0.05% aqueous solution of Dehyquat® A (Cognis). This also forms a finely dispersed precipitate.

The thus prepared, cationically stabilized particles are especially suitable for formulation into cationically based formulations for textile aftertreatment compositions, for example fabric softeners, as described in examples 8 to 10. They may either be removed by centrifugation or filtration, concentrated or else further processed directly.

### Example 5

Cationically Stabilized, Precipitated Odorant-Containing Acrylate Particles

An ethanolic solution of 1% of the acrylate polymer Ultrahold® 2 (BASF), 1% of didodecyldimethylammonium bromide (DDMABr) and 0.4% of perfume oil is prepared. A portion of this solution is introduced into 9 parts of water with stirring. A finely dispersed precipitate of polymer particles having a perfume oil content of 16.6% is obtained. As in the previous examples, the perfume-containing microparticles can be recovered by centrifugation or filtration or else be further processed directly. Owing to the cationic stabilization, this dispersion is particularly suitable for further processing in fabric softeners.

### Example 6

Precipitated Odorant-Containing Polyvinyl Acetate Particles

An ethanolic solution is prepared which contains 1% of polyvinyl acetate (Aldrich), 1% of didodecyldimethylammonium bromide (DDMABr) and 0.5% of perfume oil. A portion of this solution is introduced into 9 parts of water with stirring, in the course of which a fine precipitate of particles having a perfume oil content of 20% precipitates out. As in the previous examples, the perfume-containing microparticles can be recovered by centrifugation or filtration, or else be further processed directly. Owing to the cationic stabilization, this dispersion is particularly suitable for further processing in fabric softeners.

### Example 7

Odor Assessment

For the odor assessment, mentioned in the examples which follow, of hard surfaces, rooms or textiles a blind test is carried out in each case with a panel consisting of ten people. Each of the panel members assesses the fragrance intensity of the inventive formulations against that of the comparative example. The results reported in the examples in each case give the opinion of the majority of the panel members. In the majority of the experiments, the result was very clear.

### Example 8

Use Of The Particles From Example 1 In A Fabric Softener Formulation

5.4 g of ester quat (Stepantex® VL 90) are incorporated into 100 ml of the dispersion from example 2 (corresponding to 0.33 g of perfume oil) by heating the dispersion to 40° C. and adding the ester quat, likewise heated to 40° C., with stirring.

Comparison:

5.4 g of ester quat are, as described above, dispersed in 100 ml of water. Subsequently, 0.33 g of perfume oil is added to this dispersion.

These fabric softener formulations are subsequently tested in a washing machine: 100 ml of the particular formulations are introduced into the fabric softener compartments, so that they are metered in in the last rinse. The drum contents consist of 3.5 kg of towels.

After the washing has been removed from the machine, the test panel initially classifies the textiles rinsed with the formulation of the comparative example as more intensely fragrant. However, after one day and on each further day, the fragrance of the washing subjected to fabric softening with the inventive formulation is clearly more intense than in the comparative example.

### Example 9

Use Of The Particles From Example 2 In A Fabric Softener Formulation

5.4 g of ester quat (Stepantex® VL 90) are incorporated into 100 ml of the dispersion from example 2 (corresponding to 0.75% perfume oil) by heating the dispersion to 40° C. and adding the ester quat, likewise heated to 40° C., with stirring.

Comparison:

5.4 g of ester quat are, as described above, dispersed in 100 ml of water. Subsequently, 0.75 g of perfume oil is added to this dispersion.

These fabric softener formulations are subsequently tested as in example 8 in a washing machine. 100 ml of the particular formulations are introduced into the fabric softener compartments, so that they are metered in in the last rinse. The drum contents once again consist of 3.5 kg of towels.

After the washing has been removed from the machine, the test panel initially classifies the textiles rinsed with the formulation of the comparative example as more intensely fragrant. After one day and on each further day, the fragrance of the washing subjected to fabric softening with the inventive formulation is clearly more intense than in the comparative example.

### Example 10

Use Of The Particles From Example 4 In A Fabric Softener Formulation

The odorant-containing EC dispersion from example 4 is initially concentrated by a factor of 2 with the aid of a membrane filter having a 250 nm pore diameter. This results in a calculated perfume oil concentration of 0.6%. Small losses resulting from the filtration cannot be ruled out. 5.4 g of ester quat (Stepantex® VL 90) are incorporated into 100 ml of this dispersion (corresponding to 0.6 g of perfume oil) by heating the dispersion to 40° C. and the ester quat, likewise heated at 40° C., is added with stirring.

Comparison:

5.4 g of ester quat are, as described above, dispersed in 100 ml of water. Subsequently, 0.6 g of perfume oil is added to this dispersion.

The test is as in examples 8 and 9. Again, after the washing has been removed from the machine, the test panel initially classifies the textiles rinsed with the formulation of the comparative example as more intensely, sometimes unpleasantly intensely, fragrant. After one day and on each further day, the fragrance of the washing subjected to fabric softening with the inventive formulation was then distinctly more intense than in the comparative example.

### Example 11

Use Of The Particles From Example 5 And 6 In A Fabric Softener Formulation

These experiments are carried out in a similar manner to example 10, except that the dispersion is concentrated with the aid of the membrane filter in such a way that (calculated without taking into account possible losses) it contains 0.6% of perfume oil. The experimental procedure and the result correspond to examples 8 to 10.

### Example 12

Use Of The Particles From Example 3 In A Model Formulation Of A Cleaner For Hard Surfaces

In this example, the use of the anionically stabilized dispersion from example 3 in a model formulation for a cleaner is described. This formulation represents a model of a bathroom, kitchen, floor or all-purpose cleaner.

To this end, the dispersion from example 3 is initially concentrated with the aid of a membrane filter (pore diameter 220 nm), so that the resulting dispersion has a perfume oil content of 0.5%. To prepare the cleaner model formulation, 5 g of linear alkylbenzenesulfonate (Maranil®, Cognis) and 3 g of Dehydol® LT7 (Cognis) are added to 92 g of this dispersion. As the comparative example, 0.46 g of perfume oil is added to a surfactant solution of the same concentration. The comparative formulation itself has a more intense fragrance than the inventive formulation, and the fragrance of the inventive formulation is also masked by a slight acetone note.

For the odor assessment, these cleaner formulations (inventive and comparative example) are used to prepare a wiping solution by 10-fold dilution. This wiping solution is used, with the aid of a cloth, to wipe the PVC floor of two rooms having low air exchange. In the room in which the formulation of the comparative example is applied, the fragrance fades away even after a short time, but at the latest after ventilation. In the room which is wiped with the inventive composition, the fragrance is less intense immediately after the wiping, but it is then still perceptible over several days and slowly builds up again after ventilation.

The disclosures of each patent, patent application, and publication cited or described in this document are hereby incorporated herein by reference, in their entireties.

Various modifications of the invention, in addition to those described herein, will be apparent to those skilled in the art from the foregoing description. Such modifications are also intended to fall within the scope of the appended claims.

1. A matrix for sustained release of fragrances, comprising:
a fragrant component dispersed in a polymer having an average molecular weight of greater than 30,000, wherein the polymer is water-insoluble cellulose, a derivative of water-insoluble cellulose, or water-insoluble polymer.

2. The matrix of claim 1, wherein the matrix exhibits a sustained release effect for the fragrant component.

3. The matrix of claim 1, further comprising a surfactant.

4. The matrix of claim 1, further comprising water.

5. The matrix of claim 1, wherein the polymer has an average molecular weight of greater than 35,000.

6. The matrix of claim 1, wherein the polymer has an average molecular weight of greater than 40,000.

7. The matrix of claim 1, wherein the cellulose derivative is alkylcellulose, ethylcellulose, cellulose ester, cellulose acetate, or cellulose acetate butyrate.

8. The matrix of claim 1, wherein the polymer is a nonionic or an ionic polymer.

9. The matrix of claim 1, wherein the polymer includes at least one of a blocked co-monomer or random co-monomer.

10. The matrix of claim 1, wherein the polymer is at least one of polyalkylene, polybutadiene, poly(butadiene-co-acrylonitrile), polyisobutene, polyamide, polystyrene, polyisoprene, polycarbonate, polyester, polyacrylate, polymethacrylate, polyurethane, polydimethyl itaconate, polydiamyl fumarate, polybenzyl vinyl ether, polyvinyl alcohol, polyallyl alcohol, polyvinyl formal, polyvinyl butyral, poly(2-vinyl-4,7-dihydro-1,3-dioxepine), polyvinyl methyl ketone, polymethyl isopropyl ketone, polyvinyl pivalate, polyvinyl acetylacetate, poly(p formylstyrene), poly(2-vinylpyridine), polydimethylfulvene, poly(carbonyl-1-furfuryltrimethylene), polytetrahydrofuran, polyglutaraldehyde, polyoxycarbonyloxyhexamethylene, polyethylene adipate, polyvinyl alcohol acetate, polyvinyl butyral acetal, polylactic acid, or polyoxymethylhexadecylsilylene.

11. The matrix of claim 1, wherein the polymer has been ionically modified by grafting or by copolymerization with suitable ionic co-monomers.

12. The matrix of claim 1, wherein the polymer is an ionic polymer.

13. The matrix of claim 12, wherein the ionic polymer is an ammonium-functional (meth)acrylate copolymer, a poly(ethylene-methyl methacrylate-trimethylammonioethyl methacrylate) copolymer, optionally having stoichiometric ratios of the individual monomer units of 1:2:0.1 or 1:2:0.2.

14. The matrix of claim 1, wherein the fragrant component comprises fragrances, odorants, or oils including essential oils, aroma oils, perfume oils, care oils and silicone oils.

15. The matrix of claim 1, having an average particle size of from about 50 nm to about 500 μm.

16. The matrix of claim 1, wherein the fragrant component is from 1% by weight to 60% by weight.

17. The matrix of claim 2, wherein the surfactant is from 0.01% by weight to 50% by weight.

18. The matrix of claim 1, wherein the fragrant component is in homogeneous distribution through the polymer.

19. The matrix of claim 1, wherein the matrix has a surface charge.

20. The matrix of claim 1, wherein the matrix has a zeta potential greater than 10 mV.

21. A laundry detergent or cleaning composition comprising the matrix of claim 1.

22. A cosmetic or personal care composition comprising the matrix of claim 1.

23. A film or coating comprising the matrix of claim 1.

24. A method for producing a matrix for sustained release of fragrances, comprising:
providing a homogeneous mixture of least one polymer and at least one fragrant component with a substantially water-immiscible organic solvent or dispersant;
contacting the mixture with an aqueous solution and emulsifying the resulting mixture; and
removing the organic solvent from the emulsion to obtain fragrance-laden polymer in an aqueous dispersion.

25. The method of claim 24, further comprising at least one of stirring or heating at the step of contacting.

26. The method of claim 24, further comprising removing the fragrance laden polymer.

27. The method of claim 24, further comprising dropwise addition at the step of contacting.

28. The method of claim 24, wherein at least one of the polymer or the fragrant component are only sparingly soluble in the aqueous phase.

29. The method of claim 24, wherein the solvent or dispersant is oleic acid.

30. A method for providing sustained release of a fragrant component, comprising:
dispersing the fragrant component in a polymer having an average molecular weight of greater than 30,000 and a substantially water-immiscible organic solvent or dispersant;
emulsifying the resulting mixture along with an aqueous solution; and
removing the organic solvent from the emulsion to obtain fragrance-laden polymer in an aqueous dispersion.

31. The method of claim 30, further comprising removing the fragrance laden polymer.
